(19) United States
(12) Patent Application Publication
(10) Pub. No.: US 2023/0321329 A1
(43) Pub. Date: Oct. 12, 2023

(54) TRANSFER SETS WITH FILTERS, INCLUDING TRANSFER SETS FOR PERITONEAL DIALYSIS SYSTEMS, AND ASSOCIATED SYSTEMS, DEVICES, AND METHODS

(71) Applicant: Byonyks Medical Devices, Inc., Itasca, IL (US)

(72) Inventors: Farrukh Usman, Chatham, NY (US); Faisal Bashir, Lahore (PK); Salar Toor, Lahore (PK)

(21) Appl. No.: 18/043,504

(22) PCT Filed: Sep. 2, 2021
(86) PCT No.: PCT/US2021/048801

§ 371 Date: Feb. 28, 2023

Related U.S. Application Data
(60) Provisional application No. 63/074,496, filed on Sep. 4, 2020.

(51) Int. Cl. A61M 1/14; A61M 1/28

(52) U.S. Cl. CPC A61M 1/1522; A61M 1/155; A61M 1/285; A61M 2039/0276

(57) ABSTRACT

Transfer sets with filters, including transfer sets with filters for peritoneal dialysis (PD) systems, and associated systems, devices, and methods are disclosed herein. In one embodiment, a transfer set includes a first connector configured to be coupled to a disposable set of a PD system, a second connector configured to be coupled to a catheter of the PD system, and a fluid channel extending between the first connector and the second connector. The transfer set further includes a filter positioned within the fluid channel and configured to filter contaminants from solution flowing within the fluid channel between the first connector and the second connector. In some embodiments, the transfer set further includes a one-way valve positioned between the filter and the second connector and configured to prevent fluid from flowing through the one-way valve in a direction toward the filter.

### CROSS-REFERENCE TO RELATED APPLICATION(S)

This application is a section 371 U.S. national phase of PCT/US2021/048801,_filed Sep. 2, 2021, which claims the benefit of priority to U.S. Provisional Patent Application No. 63/074,496, filed Sep. 4, 2020, the entirety of both applications which is incorporated by reference herein.

### TECHNICAL FIELD

The present disclosure is directed to transfer sets with filters and associated systems, devices, and methods. For example, several embodiments of the present disclosure are directed to transfer sets (e.g., for peritoneal dialysis (PD) systems) that include filters configured to at least partially reduce or prevent contaminants from entering a patient fluidly coupled to the transfer set.

### BACKGROUND

Dialysis is used to (a) remove excess fluid and toxins in persons with kidney failure and (b) correct electrolyte concentrations in their blood. Peritoneal dialysis is a form of dialysis that uses a peritoneum in an individual's abdomen as a membrane through which fluid and dissolved substances are exchanged with blood. More specifically, a solution is introduced into and removed from the individual's abdomen via a surgically installed catheter.

In continuous ambulatory dialysis (CAPD), solution is manually introduced and removed (e.g., at regular intervals throughout the day). In particular, the catheter is connected to a disposable set that includes (i) a source bag (e.g., hung on a drip stand) containing new solution, (ii) a drain bag configured to collect waste solution, and (iii) various fluid lines connecting the source bag and the drain bag to the catheter. Waste solution from the individual's lower abdomen is drained into the drain bag via the catheter, and new solution is introduced into the individual's lower abdomen via the catheter. After such an exchange treatment is complete, the disposable set is discarded.

APD (also known as continuous cycling peritoneal dialysis (CCPD)) is similar to CAPD except that the exchange treatment is automated using an APD machine or cycler. More specifically, a pump included in the APD machine is used to introduce and remove the solution (e.g., while the individual sleeps). Each APD exchange treatment may include one or more cycles of introducing and removing solution from the individual's abdomen.

### BRIEF DESCRIPTION OF THE DRAWINGS

FIG. 1 is a schematic representation of a PD system configured in accordance with various embodiments of the present technology.

FIG. 2 is a partially schematic representation of a PD system including a transfer set configured in accordance with various embodiments of the present technology.

FIG. 3 is a partially schematic, perspective view of a transfer set configured in accordance with various embodiments of the present technology.

FIG. 4 is a partially schematic, cross-sectional view of a connector of a transfer set configured in accordance with various embodiments of the present technology.

FIG. 5A is a partially schematic view of a filtering connector of a transfer set configured in accordance with various embodiments of the present technology.

FIG. 5B is a partially schematic, perspective view of a filter of a filtering connector configured in accordance with various embodiments of the present technology.

FIG. 5C is a partially schematic, enlarged view of a portion of a filtering element of the filter of FIG. 5B.

FIG. 6 is a partially schematic, perspective view of a portion of a filtering element assembly configured in accordance with various embodiments of the present technology.

FIGS. 7A and 7B are partially schematic representations of a system that includes a transfer set configured in accordance with various embodiments of the present technology.

FIG. 8 is a partially schematic representation of the system of FIGS. 7A and 7B and a fluid delivery device configured in accordance with various embodiments of the present technology.

FIG. 9 is a flow diagram illustrating a method of operating a PD system including a transfer set configured in accordance with various embodiments of the present technology.

### DETAILED DESCRIPTION

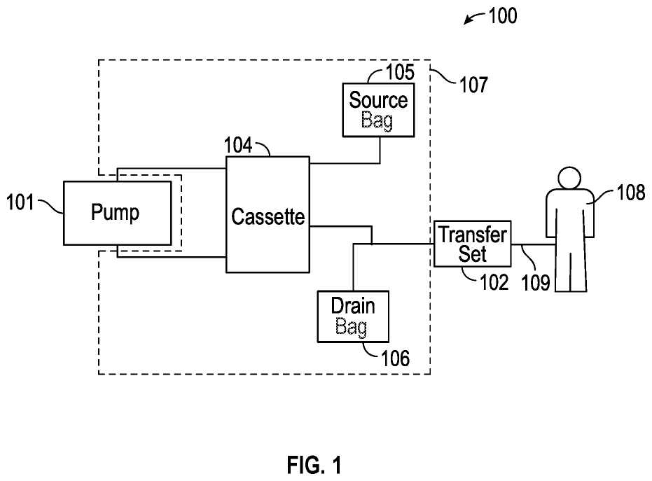

The present disclosure is directed to transfer sets with filters and associated systems, devices, and methods. In the illustrated embodiments herein, transfer sets configured in accordance with the present technology are primarily described in the context of filtering contaminants from dialysate solution flowing through a PD system. Transfer sets with filters configured in accordance with various embodiments of the present technology can be incorporated into and/or used by APD systems, CAPD systems, hemodialysis systems, and/or other medical or non-medical systems. Additionally, transfer sets configured in accordance with the present technology can be used to filter contaminants from other solutions or fluids besides dialysate solution, such as water, saline, blood, and/or other low viscous fluids. Furthermore, a person skilled in the art will understand (i) that the technology may have additional embodiments than those illustrated and described herein with reference to FIGS. 1-9 and (ii) that the technology may be practiced without several of the details of the embodiments described herein with reference to FIGS. 1-9.

### A. OVERVIEW

Patients receiving PD treatment may contract infections, such as bacterial peritonitis, during therapy. Bacterial peritonitis can be caused by foreign bacteria that are introduced into a patient's peritoneum via the patient's PD system, such as via tubing, connectors, and/or other elements associated with the PD system. Bacterial peritonitis can cause inflammation in the peritoneum during dialysis which can lead to swelling of the peritoneum. As a result, patients that contract peritonitis typically experience symptoms such as abdominal tenderness and vomiting. In some instances, or if left undetected, peritonitis can be fatal. Additionally, viruses, fungi, and other contaminants or matters can enter PD systems and can create generally similar health problems for patients receiving PD therapy. Many PD systems lack measures to prevent contamination, which can limit patient access to PD therapy. In developing countries, for example, the use of PD systems has been affected because many PD systems can be prone to frequent contamination from the environment rendering use of a PD system relatively unsafe or risky.

Contaminants can enter PD systems through openings, ports, or anywhere a connection between elements of a PD system is interrupted. For example, contaminants can enter a transfer set of a PD system when a connection between the transfer set and a disposable set is interrupted (e.g., a seal is breached). Contaminants can also enter PD systems during therapy. For example, a user may accidentally connect a transfer set to a contaminated dialysate line, which can allow contaminants to enter a patient through the transfer set and can contaminate the patient's peritoneal cavity. As another example, waste solution drained from the patient can flow into an inlet of the transfer set (as opposed to into a drain bag) and/or can backflow into the patient. Any of these events can result in a patient being infected by the contaminants which, as described previously, can result in serious health concerns.

To address these and other concerns, the inventors have developed transfer sets with filters, including transfer sets for PD systems, and associated systems, devices, and methods that are expected to safely, reliably, and affordably provide PD therapy to patients and at least partially reduce the risk of introducing contaminants into the patients. In some embodiments, a transfer set of the present technology includes (a) a first branch configured to be coupled to a source bag of a disposable set of PD system, (b) a second branch configured to be coupled to a drain bag of the disposable set, and (c) a third branch configured to be coupled to a catheter that can be at least partially surgically installed within a patient. The transfer set can be configured such that a first solution (e.g., a dialysate solution) from the source bag can flow along an inlet path (e.g., through the first branch and the third branch) of the transfer set to the catheter, and a second solution (e.g., waste solution) from the catheter can flow along an outlet path (e.g., through the third branch and the second branch) of the transfer set toward the drain bag.

In some embodiments, the first branch can include a filter configured to remove contaminants or other matters from the first solution as the first solution flows along the first branch toward the third branch. For example, the first branch can include a connector configured to facilitate fluidly coupling the first branch to the source bag, and the connector can include the filter. The filter can include one or more filtering elements (e.g., semi-permeable membranes, or any other suitable filtering elements). In at least some embodiments, for example, individual ones of the one or more filtering elements include a semipermeable membrane configured to (i) permit the first solution to flow through the semi-permeable membrane and continue flowing toward the catheter and (ii) prevent contaminants or other matters from flowing through the semi-permeable membrane.

In some embodiments, the first branch of the transfer set includes an injection port. The injection port can be positioned proximate the filter and/or between the filter and the catheter. The injection port can be configured to be removably coupled to a fluid delivery device and to receive a fluid from the fluid delivery device. As the fluid is injected into the injection port from the fluid delivery device, the fluid can flow across the filter in a direction generally opposite to the direction the first solution flows from the source bag through the filter. As a result, the fluid can flush contaminants from the filter or otherwise clean or unclog the filter.

In these and other embodiments, the first branch of the transfer set includes a one-way valve positioned between the filter and the catheter. The one-way valve of the first branch can be configured to at least partially prevent fluid from flowing through the one-way valve toward the filter. As a result, when the transfer set is used to transfer the second solution received from the catheter toward the drain bag, the one-way valve of the first branch can prevent the second solution from flowing along at least a portion of the first branch and/or from contaminating the filter or other components of the transfer set positioned along the first branch.

In some embodiments, the second branch of the transfer set includes a one-way valve positioned between the drain bag and the catheter. The one-way valve of the second branch can be configured to at least partially prevent fluid from flowing through the one-way valve toward the catheter or the first branch. As a result, when the transfer set is used to transfer the second solution received from the catheter toward the drain bag, the one-way valve of the second branch can prevent the second solution from flowing into the first branch and/or from backflowing into the catheter.

Therefore, transfer sets of the present technology can be configured to (a) filter contaminants (e.g., bacteria, viruses, fungi, etc.) or other matters from dialysate solution flowing through the transfer set and (b) restrict a direction of flow of at least waste solution flowing through the transfer set. As a result, the transfer sets of the present technology are expected to at least partially reduce or prevent introducing contaminants (e.g., bacteria, viruses, fungi, or other matters) into the patient. In turn, this is expected to reduce or prevent the risk of patient infections stemming from use of a PD system.

### B. SELECTED EMBODIMENTS OF CATHETER EXTENSION TUBE FILTER FOR PERITONEAL DIALYSIS SYSTEMS, AND ASSOCIATED SYSTEMS, DEVICES, AND METHODS

FIG. 1 is a schematic representation of a PD system 100 (“the system 100”) configured in accordance with various embodiments of the present technology. The PD system 100 can be part of an APD system, a CAPD system, or any other suitable PD system. As shown, the system 100 includes a disposable set 107, a transfer set 102, a catheter 109, and a pump 101. In other embodiments, the system 100 can omit the pump 101. Other well-known components of PD systems are not illustrated in FIG. 1 or described in detail below so as to avoid unnecessarily obscuring aspects of the present technology.

The disposable set 107 of FIG. 1 includes a cassette 104, a source bag 105, a drain bag 106, and various fluid lines extending between components of the disposable set 107 and/or other components of the system 100. For example, the disposable set 107 can be configured to interface (i) with the pump 101, (ii) with the transfer set 102, and/or (iii) with the catheter 109. As a more specific example, the disposable set 107 can connect to the catheter 109 (e.g., directly or via the transfer set 102) such that the catheter 109 is placed in fluid communication with the source bag 105 and/or the drain bag 106. In some embodiments, the transfer set 102 can be disposable and/or can be part of the disposable set 107.

In some embodiments, the pump 101 can be a peristaltic pump or another suitable type of pump in which fluid is isolated from pumping mechanisms. In these and other embodiments, the pump 101 can be removably or permanently integrated into an APD machine. Alternatively, the pump 101 can be a component ofthe system 100 that is separate from the APD machine. As discussed above, various components of the disposable set 107 can interface with an APD machine and/or a CAPD stand or assembly. For example, a portion of the drain bag 106 can be mounted or otherwise positioned on an APD machine and/or a CAPD stand or assembly.

In some embodiments, the transfer set 102 includes one or more fluid lines that serve as an extension of the catheter 109 and facilitate fluidly coupling the catheter 109 to various components of the disposable set 107. For example, the transfer set 102 can direct or control fluid flow (a) between the source bag 105 of the disposable set 107 and the catheter 109 and/or (b) between the catheter 109 and the drain bag 106 of the disposable set 107. As described in greater detail below, the transfer set 102 can additionally, or alternatively, filter one or more contaminants (e.g., bacteria, viruses, infection-causing and/or disease-causing material, etc.) from fluid flowing through the transfer set 102. For example, the transfer set 102 (i) can include a contaminant-filtering connector positioned between the source bag 105 and the catheter 109, and (ii) can filter solution from the source bag 105 as the solution flows from the source bag 105 through the filtering connector of the transfer set 102 on its way to the catheter 109 (e.g., for introduction into a patient 108). In some embodiments, the transfer set 102 can be multi-use or reusable. For example, the transfer set 102 (a) can be at least partially disconnected from the disposable set 107 and/or the catheter 109 and/or (b) can be cleaned or sterilized (e.g., “flushed”) to at least partially remove contaminants from the transfer set 102 (e.g., from the contaminant-filtering connector of the transfer set 102). In these and other embodiments, the transfer set 102 can be single-use or disposable.

In operation, the system 100 can be configured to introduce solution (e.g., dialysate or another fluid initially contained within the source bag 105) into the patient 108 using the pump 101 and/or via at least a first portion of the disposable set 107 and the transfer set 102. The system 100 can further be configured to remove solution from the patient 108 by draining the solution (e.g., waste solution) into the drain bag 106 using the pump 101 and/or via the transfer set 102 and at least a second portion of the disposable set 107. In some embodiments, a single exchange treatment can include one or more cycles of introducing solution into the patient 108 and removing solution from the patient 108. After an exchange treatment is complete, the disposable set 107 and/or the transfer set 102 can be discarded and a separate (e.g., a new) disposable set 107 and/or transfer set 102 can be used for a subsequent treatment. For example, the disposable set 107 can be discarded, the transfer set 102 can be cleaned or sterilized, and a new disposable set 107 can be connected to the transfer set 102 for a subsequent treatment. As another example, both the disposable set 107 and the transfer set 102 can be discarded, a new transfer set 102 can be connected to the catheter 109, and a new disposable set 107 can be connected to the transfer set 102 for a subsequent treatment.

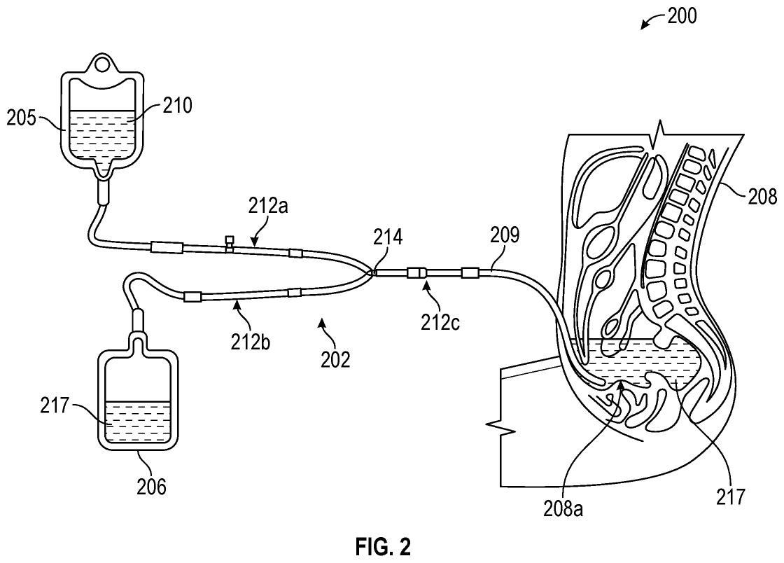

FIG. 2 is a partially schematic representation of a PD system 200 (“the system 200”) configured in accordance with various embodiments of the present technology. As shown, the system 200 includes a transfer set 202, a source bag 205, a drain bag 206, and catheter 209. The system 200 can be at least a portion of the system 100 of FIG. 1, or at least a portion of another PD system configured in accordance with various embodiments of the present technology. Accordingly, one or more components of the system 200 (e.g., the transfer set 202, the source bag 205, etc.) can be components of the system 100 (e.g., the transfer set 102, the source bag 105, etc.), or of another PD system configured in accordance with the present technology.

The transfer set 202 can include one or more branches 212 configured to transfer or otherwise allow fluid to flow between the source bag 205, the patient 208 (e.g., a peritoneum or peritoneal space 208a of the patient), and/or the drain bag 206. In the illustrated embodiment, for example, the transfer set 202 includes a first (e.g., inlet, inflow) branch 212a fluidly coupled to the source bag 205, a second (e.g., outlet, outflow) branch 212b fluidly coupled to the drain bag 206, and a third branch 212c fluidly coupled to the catheter 209. Each of the branches 212 can be coupled (e.g., operably coupled, fluidly coupled, etc.) to one another via a hub or splitter 214. The splitter 214 can be configured such that fluid from one of the branches 212 (e.g., the first branch 212a) can flow to at least one of the other branches 212 (e.g., the second branch 212b and/or the third branch 212c). Accordingly, the branches 212 and the splitter 214 can define one or more fluid paths, channels, or routes through the transfer set 202.

As shown in FIG. 2, the first branch 212a and the third branch 212c of the transfer set 202 are configured to receive solution 210 (e.g., dialysate solution) from the source bag 205 and transfer (e.g., deliver, administer, transport, etc.) the solution 210 to the catheter 209 for delivery of the solution 210 into the peritoneal space 208a of the patient 208. As described in greater detail below, the first branch 212a can include a contaminant-filtering connector configured to at least partially remove one or more contaminants from the solution 210 before the solution 210 is introduced into the patient 208. Additionally, or alternatively, the third branch 212c and the second branch 212b of the transfer set 202 are configured to drain solution 217 (e.g., waste solution) from the peritoneal space 208a of the patient 208 via the catheter 209 into the drain bag 206.

Although the transfer set 202 in FIG. 2 is illustrated as having three branches 212a-c, in other embodiments the transfer set 202 can have more or fewer branches 212. In at least some embodiments, for example, the transfer set 202 can have one, three, four, five, or any other suitable number of branches. In the embodiment illustrated in FIG. 2, the splitter 214 is a bifurcated or Y-shaped splitter. In other embodiments, however, the splitter 214 can be any other suitable splitter. In some embodiments, for example, the configuration of the splitter 214 can correspond to the number of branches 212 of the transfer set 202. For example, the splitter 214 can be a Y-shaped splitter when the transfer set 202 has three branches 212, the splitter 214 can be an X-shaped or trifurcated splitter when the transfer set 202 has four branches 312, etc.

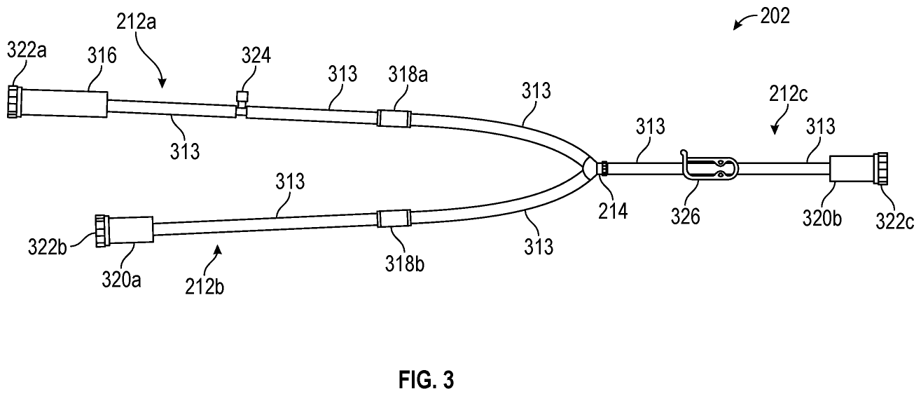

FIG. 3 is a partially schematic, perspective view of the transfer set 202 of FIG. 2. As shown in FIG. 3, the branches 212 of the transfer set 202 can include one or more elements or components that affect fluid flow through the transfer set 202. For example, the first branch 212a can include a contaminant-filtering connector 316 (“the filtering connector 316”), one or more injection ports 324, and a one-way valve 318a (e.g., a flow-biasing element); the second branch 212b can include a connector 320a and a one-way valve 318b; and the third branch 212c can include one or more clamps 326 (e.g., flow-restricting members) and a connector 320b. The various elements of the branches 212 can be coupled (e.g., operably coupled, fluidly coupled, etc.) and/or interconnected by one or more fluid lines 313 or segments. Each coupling between the various elements of the branches 212 and the fluid lines 313 can form an impermeable or hermetic seal such that fluid is prevented from leaking from the transfer set 202 (e.g., at least at locations along the transfer set 202 corresponding to the couplings) and/or such that contaminants or other matters are prevented from entering the transfer set 202 (e.g., at least at locations along the transfer set 202 corresponding to the couplings).

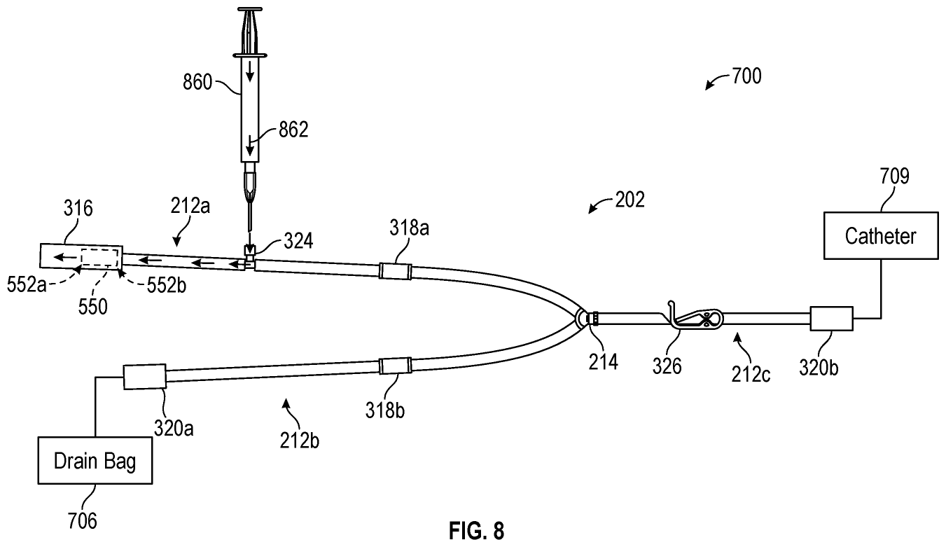

The injection port 324 of FIG. 3 is positioned proximate the filtering connector 316 and between the filtering connector 316 and the one-way valve 318a of the first branch 212a. As described in greater detail below with respect to FIG. 8, the injection port 324 can be configured to receive a fluid (e.g., a cleaning or sterilizing fluid) from an external fluid delivery device (not shown in FIG. 3), such as a syringe. More specifically, the injection port 324 can include a valve or an impermeable or hermetic seal. Fluid can be injected into the transfer set 202 through the valve or seal when the valve or seal interfaces with a fluid delivery device. Otherwise, the valve or seal can remain closed such that fluid, contaminants, or other matters are prevented from entering the transfer set 202 via the injection port 324. Fluid received through the injection port 324 can be used to at least partially clean or sterilize a portion of the transfer set 202, such as a filter (not shown in FIG. 3) included in the filtering connector 316.

As discussed in greater detail below, the filtering connector 316 and the connectors 320a, 320b can be used to couple (e.g., operably couple, fluidly couple, etc.) respective branches 212 of the transfer set 202 to components of a disposable set (e.g., the disposable set 107 of FIG. 1) and/or a catheter (e.g., the catheter 109 and/or 209 of FIGS. 1 and/or 2). For example, the filtering connector 316 can be configured to couple the first branch 212a of the transfer set 202 to a first portion of a disposable set (not shown) such that the first branch 212a is coupled to a source bag (not shown) of the disposable set. As another example, the connector 320a can be configured to couple the second branch 212b of the transfer set 202 to a second portion of a disposable set such that the second branch 212b is coupled to a drain bag (not shown) of the disposable set. As still another example, the connector 320b can be configured to couple the third branch 212c of the transfer set 202 to a catheter (not shown), such as a catheter surgically installed in a patient.

As shown in FIG. 3, the filtering connector 316, the connector 320a, and/or the connector 320b can include a respective end cap 322a-c or cover (“the end caps 322”). Each of the end caps 322 can be releasably coupled to the respective filtering connector 316, connector 320a, or connector 320b at least when the respective filtering connector 316, connector 320a, or connector 320b is not coupled to a disposable set or to a catheter. In some embodiments, the end caps 322 can include O-rings or other suitable sealing elements. Thus, when the end caps 322 are releasably coupled to the filtering connector 316, the connector 320a, and/or the connector 320b, the end caps 322 can be configured to form an impermeable or hermetic seal with the respective filtering connector 316, connector 320a, or connector 320b. The impermeable or hermetic seal can prevent fluid, contaminants, or other matters from entering the filtering connector 316, the connector 320a, and/or the connector 320b of the transfer set 302.

Although elements of the transfer set 302 of FIG. 3 are illustrated as having specific positions or orientations relative to each other, the elements can be rearranged and/or have any other suitable position or orientation relative to the other elements of the transfer set 302 in other embodiments of the present technology. Additionally, or alternatively, one or more elements illustrated in FIG. 3 can be omitted in other transfer sets configured in accordance with various embodiments of the present technology. For example, the injection port 324, the one-way valve 318a, the one-way valve 318b, and/or the clamp 326 can be omitted in some embodiments. As another example, the transfer set 302 can include a connector (e.g., similar to the connector 320b) that lacks a filter in lieu of the filtering connector 316. In these and other embodiments, the transfer set 202 can include a clamp at other locations in addition to, or in lieu of, the clamp 326 in the third branch 212c. For example, the first branch 212a of the transfer set 202 can include a clamp between the filtering connector 316 and the splitter 214, such as between the injection port 324 and the one-way valve 318a, or at any other suitable position on the first branch 212a. Additionally, or alternatively, the second branch 212b of the transfer set 202 can include a clamp positioned between the connector 320a and the splitter 214, such as between the connector 320a and the one-way valve 318b or between the one-way valve 318b and the splitter 214, or at any other suitable position on the second branch 212b. In these and other embodiments, the transfer set 202 can include a filter at other locations in addition to, or in lieu of, the filter in the filtering connector 316. For example, the transfer set 202 can include a filter after the filtering connector 316, such as in the first branch 212a between the filtering connector 316 and the injection portion 324, between the filtering connector 316 and the one-way valve 318a, and/or between the one-way valve 318a and the splitter 214. As further examples, the transfer set 202 can include a filter in the splitter 214 and/or in the third branch 212c (e.g., in the connector 320b). Continuing with the above examples, the transfer set 202 can include one or more injection ports 324 at locations proximate (e.g., immediately downstream) each of the filters.

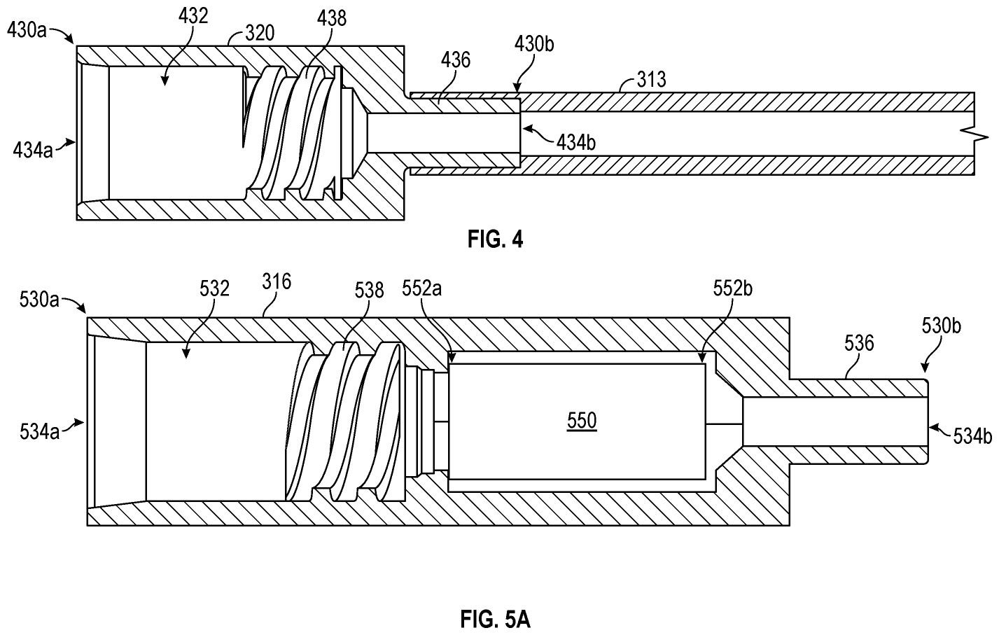

FIG. 4 is a partially schematic, cross-sectional view of a connector 320 (e.g., the connector 320a or the connector 320b) of the transfer set 202 of FIGS. 2 and 3. As shown, the connector 320 includes a first end portion 430a, a second end portion 430b opposite the first end portion 430a, and a fluid channel 432 extending between the first end portion 430a and the second end portion 430b. More specifically, during operation, fluid can enter the connector 320 though a first opening 434a or port at the first end portion 430a, travel along the channel 432 within the connector 320, and exit the connector 320 (e.g., into a fluid line 313 coupled to the connector 320) through a second opening 434b or port at the second end portion 430b. Alternatively, fluid can enter the connector 320 (e.g., from the fluid line 313) through the second opening 434b at the second end portion 430b, travel along the channel 432, and exit the connector 320 through the first opening 434a at the first end portion 430a.

As discussed above, the connector 320 can be releasably coupled to an end cap 322 (FIG. 3). In particular, the first end portion 430a and/or the first opening 434a of the connector 320 can releasably receive the end cap 322 to form an impermeable or hermetic seal. The seal formed at the first end portion 430a of the connector 320 using the end cap 322 can prevent fluid, contaminants, or other matters from entering into the connector 320 via the opening 434a at the first end portion 430a of the connector 320.

In these and other embodiments, with the end cap 322 uncoupled from the first end portion 430a of the connector 320, the first end portion 430a and/or the first opening 434a can releasably receive (a) a corresponding connector (not shown) or adapter of a disposable set (e.g., to couple the connector 320 to a drain bag of the disposable set, such as the drain bag 106 and/or 206 of FIGS. 1 and/or 2), and/or (b) a corresponding connector (not shown) or adapter of a catheter (e.g., the catheter 109 and/or 209 of FIGS. 1 and/or 2). In some embodiments, the fluid channel 432 of the connector 320 can include a retention element 438. The retention element 438 can include threading, one or more fasteners, one or more latches, one or more clasps, one or more adhesives, and/or any other suitable retention feature(s). In operation, the retention element 438 can securely and releasably couple the connector 320 to a corresponding connector of a disposable set or a catheter (e.g., by retaining at least a portion of the connector of the disposable set or the catheter within the connector 320) or to an end cap 322. It is expected that a connector 320 including a retention element 438 configured in accordance with various embodiments of the present technology can exhibit a reduced risk of accidental and/or unintentional decoupling of the connector 320 with a connector of a disposable set or a catheter or with an end cap 322, which, in turn, can at least partially reduce the risk of contaminants entering the connector 320 via the first opening 434a. In some embodiments, the first end portion 430a can include an O-ring or another suitable sealing element. Thus, when coupled to a corresponding connector of a disposable set or a catheter, the first end portion 430a can at least partially form an impermeable of hermetic seal with the corresponding connector (e.g., to (a) prevent fluid, contaminants, or other matters from entering the connector 320 via the first opening 434a, and/or (b) prevent fluid from leaking out from the coupling between the connector 320 and the corresponding connector of the disposable set or the catheter).

In some embodiments, the second end portion 430b of the connector 320 can include a mating adapter 436 configured to facilitate coupling the connector 320 to the fluid line 313. In the illustrated embodiment, the mating adapter 436 is configured to be releasably received within an interior of the fluid line 313. In other embodiments, the mating adapter 436 can be configured (a) to releasably receive the fluid line 313 within an interior of the mating adapter 436 and/or (b) to interface with and couple to the fluid line 313 in another suitable manner. When coupled to the fluid line 313, the mating adapter 436 can at least in part form an impermeable or hermetic seal (e.g., to prevent fluid, contaminants, or other matters from entering the connector 320 via the second opening 434b and/or from entering the fluid line 313, and/or (b) prevent fluid from leaking out from the coupling between the mating adapter 436 and the fluid line 313.

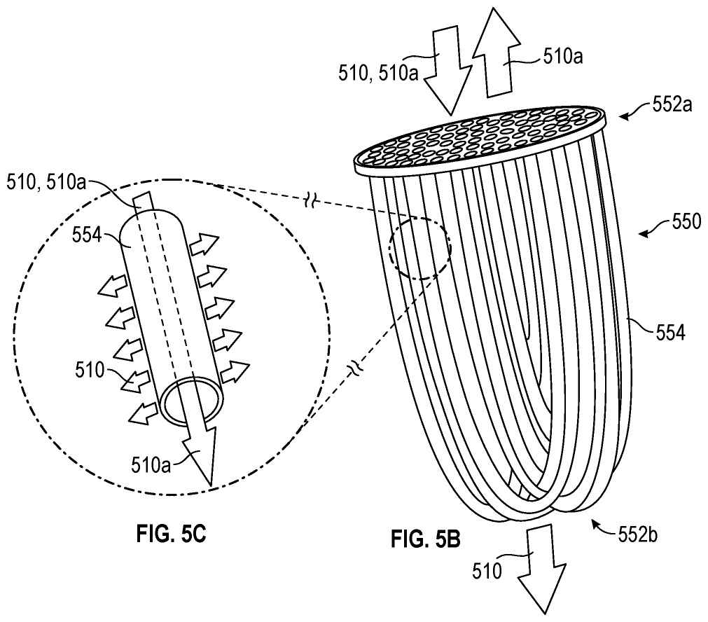

FIG. 5A is a partially schematic view of the filtering connector 316 of the transfer set 202 of FIGS. 2 and 3. As shown, the filtering connector 316 includes a first end portion 530a, a second end portion 530b opposite the first end portion 530a, and a fluid channel 532 extending between the first end portion 530a and the second end portion 530b. More specifically, fluid can enter the filtering connector 316 through a first opening 534a or port at the first end portion 530a, travel along the channel 532, and exit the filtering connector 316 (e.g., into a fluid line coupled to the filtering connector 316) through a second opening 534b or port fluidly coupled to the first opening 534a via the interior channel 532 at the second end portion 530b.

As discussed above, the filtering connector 316 can be releasably coupled to the end cap 322 (FIG. 3). In particular, the first end portion 530a and/or the first opening 534a of the filtering connector 316 can releasably receive the end cap 322 to form an impermeable or hermetic seat. The seal formed at the first end portion 530a of the filtering connector 316 using the end cap 322 can prevent fluid, contaminants, or other matters from entering into the connector 320 via the opening 534a at the first end portion 530a of the filtering connector 316.

In these and other embodiments, with the end cap 322 uncoupled from the first end portion 530a of the filtering connector 316, the first end portion 530a and/or the first opening 534a can releasably receive a corresponding connector (not shown) or adapter of a disposable set (e.g., to couple the filtering connector 316 to a source bag of the disposable set, such as the source bag 105 and/or 205 of FIGS. 1 and/or 2). In some embodiments, the fluid channel 532 of the filtering connector 316 can include a retention element 538. The retention element 538 can include threading, one or more fasteners, one or more latches, one or more clasps, one or more adhesives, and/or any other suitable retention feature(s). In operation, the retention element 538 can securely and releasably couple the filtering connector 316 to a corresponding connector of a disposable set (e.g., by retaining at least a portion of the connector of the disposable set within the filtering connector 316) or to an end cap 322. It is expected that a filtering connector 316 including a retention element 538 configured in accordance with various embodiments of the present technology can exhibit a reduced risk of accidental and/or unintentional decoupling of the filtering connector 316 with a connector of a disposable set or with an end cap 322, which, in turn, can at least partially reduce the risk of contaminants entering the filtering connector 316 via the first opening 534a. In some embodiments, the first end portion 530a can include an O-ring or another suitable sealing element. Thus, when coupled to a corresponding connector of a disposable set, the first end portion 530a can at least partially form an impermeable of hermetic seal with the corresponding connector (e.g., to (a) prevent fluid, contaminants, or other matters from entering the filtering connector 316 via the first opening 534a, and/or (b) prevent fluid from leaking out from the coupling between the filtering connector 316 and the corresponding connector of the disposable set).

In some embodiments, the second end portion 530b of the filtering connector 316 can include a mating adapter 536 configured to facilitate coupling the filtering connector 316 to a fluid line, such as a fluid line 313 of FIG. 3. In the illustrated embodiment, the mating adapter 536 is configured to be releasably received within an interior of a fluid line. In other embodiments, the mating adapter 536 can be configured (a) to releasably receive a fluid line within an interior of the mating adapter 536 and/or (b) to interface with and couple to a fluid line in another suitable manner. When coupled to a fluid line, the mating adapter 536 can at least in part form an impermeable or hermetic seal (e.g., to prevent fluid, contaminants, or other matters from entering the filtering connector 316 via the second opening 534b and/or from entering the fluid line, and/or (b) prevent fluid from leaking out from the coupling between the mating adapter 536 and the fluid line.

The filtering connector 316 can include one or more filters. For example, as shown in FIG. 5A, the filtering connector 316 includes a filter 550. The filter 550 has a first end portion 552a and a second end portion 552b opposite the first end portion 552a. The first end portion 552a of the filter 550 is positioned proximate the first end portion 530a of the filtering connector 316 (e.g., proximate the retention element 538), and the second end portion 552b of the filter 550 is positioned proximate the second end portion 530b of the filtering connector 316. The filter 550 can be generally or substantially aligned with a longitudinal axis of the filtering connector 316. Accordingly, fluid can enter the filtering connector 316 via the first opening 534a, pass through the filter 550, and exit the filtering connector 316 via the second opening 534b.

FIG. 5B is a partially schematic perspective view of the filter 550 of FIG. 5A, and FIG. 5C is a partially schematic, enlarged view of a portion of a filtering element 554 of the filter 550 of FIG. 5B. Referring to FIGS. 5B and 5C together, the filter 550 can include one or more filtering elements 554 that extend at least partway between the first end portion 552a and the second end portion 552b of the filter 550. In the illustrated embodiment, each of the filtering elements 554 can be elongate and/or generally curved or arcuate, such that both ends of the filtering elements 554 can be coupled to a coupling element 556 (e.g., a disk or plate) positioned at the first end portion 552a of the filter 550. Each of the filtering elements 554 can include a semipermeable membrane formed at least partially from one or more polymers, such as polypropylene, polysulfone, polyethersulfone (PES), polyvinylidene difluoride (PVDF, PVF₂, or polyvinylidene fluoride), polyacrylonitrile (PAN), and/or any other suitable material. For example, all or a portion of the sidewalls of the filtering elements 554 can include pores (not shown) and serve as at least a portion of the semipermeable membrane. In some embodiments, the sizes (e.g., widths, diameters) of the pores can be between about 0.001 μm and about 10 μm, such as between about 0.01 μm and about 0.1 μm, or any other suitable size. In some embodiments, the filter 550 can be an ultrafilter (UF), such as an ultrafilter manufactured by Synder Filtration, Inc. of Vacaville, California, or any other suitable ultrafilter. In other embodiments, the sizes of the pores can be smaller than 0.001 μm or greater than 10 μm, or the filter 550 can be another suitable type of filter, such as a nanofilter or a microfilter.

In operation, solution 510 can enter the filter 550 at the first end portion 552a of the filter 550 via an opening at an end of a filtering element 554 of the filter 550. The solution 510 can pass through pores (e.g., in the sidewalls) of the filtering element 554 (e.g., via diffusion, osmosis, etc.) and exit the filter 550 at the second end portion 552b of the filter 550. In the event that the solution 510 includes contaminants 510a or other matters, the filtering element 554 can prevent the contaminants 510a or other matters from passing through the pores of the filtering element 554. As a specific example, hydrostatic pressure can at least partially drive the solution 510 through the filter 550 and/or the filtering elements 554. The contaminants 510a or other matters can be trapped in the filtering elements 554 (e.g., proximate the second end portion 552b of the filter 550) and/or can be pushed out of the other end of the filtering elements 554. Accordingly, the filter 550 can at least partially remove (e.g., filter, separate, etc.) the contaminants 510a or other matters from the solution 510 while allowing the solution 510 to flow through the filter 550 (and proceed into the transfer set 202 of FIG. 3). In some embodiments, as described in greater detail below with respect to FIG. 8, fluid can flow through the filter 550 in a reverse direction from the second end portion 552b toward the first end portion 552a (e.g., from outside of the filtering elements 554 to inside the filtering elements 554 via diffusion, osmosis, etc.), for example, to at least partially remove or flush the contaminants 510a out of the filtering elements 554 (e.g., via openings at ends of the filtering elements 554 at the first end portion 552a of the filter 550).

Although the filter 550 in FIG. 5B is illustrated as having one or more curved or arcuate filtering elements 554 that extend at least partway between the first end portion 552a and the second end portion 552b of the filter 550, the filter 550 can include one or more filtering elements 554 with other shapes, structures, positions, orientations, and/or other properties in other embodiments. For example, the filter 550 can include one or more filtering elements that are at least partially planar. The planar filtering element(s) can be positioned or oriented generally perpendicular to the direction of fluid flow through the filter 550 and/or the filtering connector 316. For example, the planar filtering element(s) can be positioned or oriented within a fluid channel and/or such that (i) a first side (e.g., a first surface) of the planar filtering element(s) faces toward the first end portion 552a of the filter 550 and/or (ii) a second side (e.g., a second surface) of the planar filtering element(s) faces toward the second end portion 552b of the filter 550. The planar filtering element(s) can be positioned at the first end portion 552a of the filter 550, at the second end portion 552b of the filter 550, and/or at a location between the first end portion 552a of the filter 550. In these and other embodiments, the planar filtering element(s) can extend at least partway between the first end portion 552a and the second end portion 552b. For example, the planar filtering elements can be aligned (e.g., layered, stacked, etc.) and/or positioned generally parallel to one another. The planar filtering element(s) can have filtering properties (e.g., material(s), pore size(s), semi-permeable membrane(s), etc.) that are generally similar to or the same as the filtering properties of the filtering elements 554 described in detail above. Thus, in operation, the planar filtering element(s) can function generally similar to the filtering elements 554.

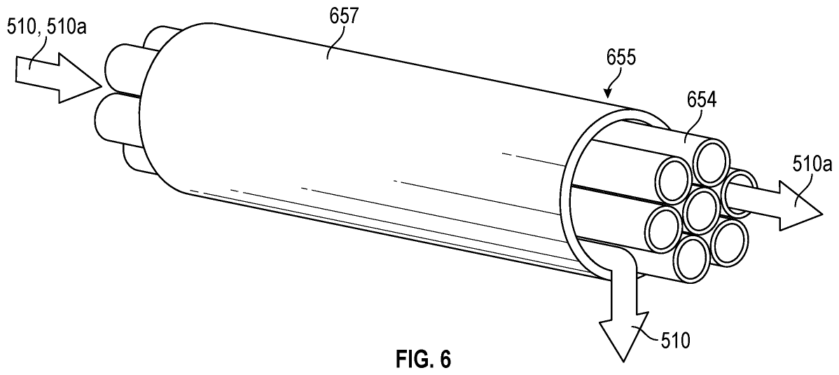

FIG. 6 is a partially schematic perspective view of a filtering element assembly 655 configured in accordance with various embodiments of the present technology. As shown, the filtering element assembly 655 includes one or more filtering elements 654 that can be generally similar to or the same as the filtering elements 554 of FIGS. 5B and 5C. The filtering element assembly 655 can further include a conduit 657 or housing configured to contain the one or more filtering elements 654. In some embodiments, the conduit 657 can be generally similar to or the same as the filtering elements 554 and/or 654. For example, the conduit 657 can (a) allow solution 510 to pass through pores (e.g., in sidewalls) of the conduit 657 (e.g., via diffusion, osmosis, etc.) and/or (b) prevent contaminants 510a or other matters from passing through the pores. The pores can have sizes similar to or different from (e.g., smaller or larger than) the pores of the filtering elements 554 and/or 654. In these embodiments, the conduit 657 can provide a second stage of filtering after a first stage of filtering provided by the filtering elements 654. In other embodiments, the conduit 657 can be impermeable to the solution 510, and/or can be provided to contain and/or direct solution 510 that exits the filtering elements 654.

In some embodiments, the filter 550 (FIGS. 5A-5C) can include one or more filtering element assemblies 655 in addition to or in lieu of the individual filtering elements 554 (FIGS. 5B and 5C). In other embodiments, the filtering elements 554 of the filter 550 of FIGS. 5B and 5C can be the filtering elements 654 of FIG. 6 and can be organized into one or more filtering element assemblies 655. In still other embodiments, the filter 550 can omit filtering element assemblies 655.

Although the filtering element assembly 655 of FIG. 6 is illustrated as having seven filtering elements 654 positioned with the conduit 657, in other embodiments the filtering element assembly 655 can include more or fewer filtering elements 654 positioned within the conduit 657. For example, in other embodiments the filtering element assembly 655 can include one, two, three, four, five, six, eight, nine, ten, or any other suitable number of filtering elements 654 positioned within the conduit 657. Furthermore, although the filtering connector 316 is discussed above and illustrated in FIGS. 5A-6 as having a single filter 550, filtering connectors 316 configured in accordance with other embodiments of the present technology can include more than one filter 550. For example, a filtering connector 316 can include two, three, four, or any other suitable number of filters 550.

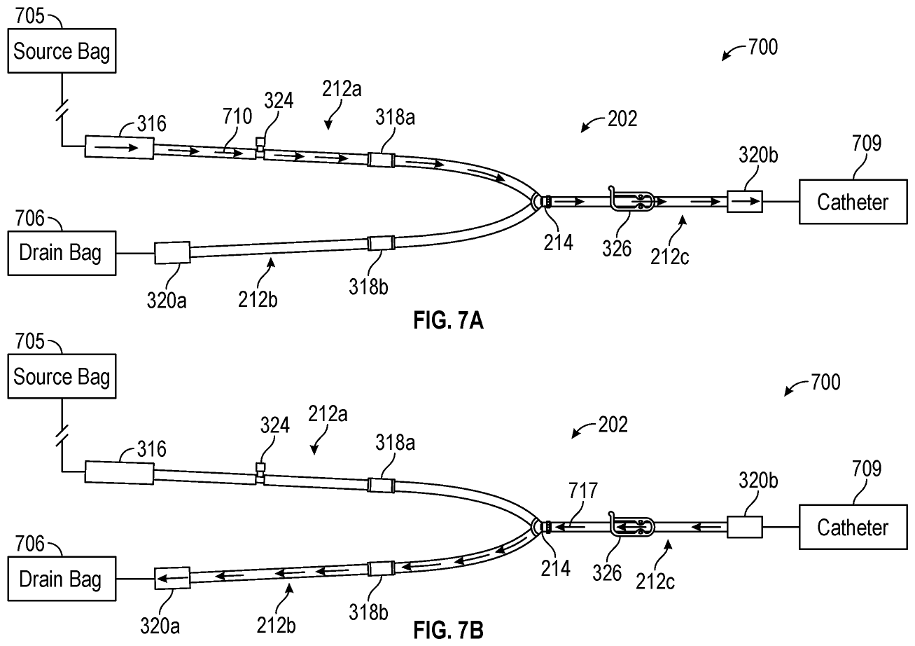

FIGS. 7A and 7B are partially schematic representations of a system 700 that includes the transfer set 202 of FIGS. 2 and 3. As shown, the filtering connector 316 of the transfer set 202 is coupled to a source bag 705 (e.g., the source bag 105 of FIG. 1, the source bag 205 of FIG. 2, or another source bag configured in accordance with the present technology) of a disposable set. In addition, the connector 320a is coupled to a drain bag 706 (e.g., the drain bag 106 of FIG. 1, the drain bag 206 of FIG. 2, or another drain bag configured in accordance with the present technology) of the disposable set. Furthermore, the connector 320b is coupled to a catheter 709 (e.g., the catheter 109 of FIG. 1, the catheter 209 of FIG. 2, or another catheter configured in accordance with the present technology). In some embodiments, the catheter 709 can be surgically installed within a patient (not shown).

FIG. 7A illustrates the transfer set 202 in operation while introducing solution 710 from the source bag 705 into a patient (not shown) via the catheter 709. As shown, the first branch 212a can be configured to receive the solution 710 from the source bag 705 via the filtering connector 316. In accordance with the discussion of FIGS. 5A-6 above, as the solution 710 passes through the filtering connector 316, a filter (not shown in FIG. 7A) of the filtering connector 316 can remove contaminants or other matters from the solution 710 and prevent the contaminants or other matters from exiting the filtering connector 316 and proceeding along the first branch 212a of the transfer set 202. After exiting the filtering connector 316, the solution 710 flows (a) through the injection port 324 and the one-way valve 318a and (b) into the third branch 212c of the transfer set 202 via the splitter 214. Once the solution 710 passes through the one-way valve 318a, the one-way valve 318a can prevent the solution 710 from flowing in an opposite direction back toward the injection port 324 and/or the filtering connector 316. As shown, the clamp 326 on the branch 212c of the transfer set 202 is in an open state or configuration. Thus, after entering the third branch 212c of the transfer set 202 via the splitter 214, the solution 710 proceeds to exit the transfer set 202 via the connector 320b and pass into the catheter 709 (e.g., for introduction into the patient). As discussed previously regarding FIG. 3, the second branch 212b can include a clamp (not shown), such as a clamp similar to the clamp 326. The clamp can be placed in a closed state or configuration such that a portion of the solution 710 that passes through the one-way valve 318a is at least partially blocked or otherwise prevented from reaching the drain bag 706. In turn, the portion of the solution 710 can be redirected into the third branch 212c of the transfer set 202 and/or can exit the transfer set 202 (e.g., into the catheter 709) via the connector 320b.

FIG. 7B illustrates the transfer set 202 in operation while draining solution 717 (e.g., waste solution) from a patient (not shown) via the catheter 709 and into the drain bag 706. As shown, the third branch 212c can be configured to receive the solution 717 from the catheter 709 via the connector 320b. The clamp 326 on the branch 212c of the transfer set 202 is in the open state or configuration. In embodiments in which the second branch 212b includes a clamp, the clamp on the second branch can be placed in an open state or configuration such that the waste solution 717 can flow through the clamp and/or such that the clamp does not block or otherwise prevent the waste solution 717 from reaching the drain bag 706. Thus, after entering the third branch 212c of the transfer set 202 via the connector 320b, the solution 717 can proceed (a) into the second branch 212b of the transfer set 202 via the splitter 214, (b) through the one-way valve 318b, and (c) into the drain bag 706 via the connector 320a. In some embodiments, a portion of the solution 717 may flow into the first branch 212a of the transfer set 202 instead of flowing into the second branch 212b. In these embodiments, the one-way valve 318a of the first branch 212a can prevent the solution 717 from proceeding further along the first branch 212a beyond the one-way valve 318a. This can reduce the likelihood that contaminants or other matters (e.g., in the solution 717 received from the catheter 709) contact or otherwise interact with the various components (e.g., the filtering connector 316, the injection port 324, and/or the fluid lines 313 (FIG. 3)) of the first branch 212a of the transfer set 202. Accordingly, in at least some embodiments, the one-way valve 318a can ensure that all or substantially all the solution 717 that enters the transfer set 202 via the catheter 709 drains into the drain bag 706 via the second branch 212b. Additionally, or alternatively, once the solution 717 passes through the one-way valve 318b of the second branch 212b of the transfer set 202, the one-way valve 318b can prevent the solution 717 from flowing in an opposite direction back toward the splitter 214. This can reduce the likelihood that contaminants or other matters (e.g., from the drain bag 706 or included in solution 717) proceed into the transfer set 202 beyond the one-way valve 318b.

FIG. 8 is a partially schematic representation of the system 700 of FIGS. 7A and 7B with the filtering connector 316 uncoupled form the source bag 705 (FIGS. 7A and 7B). In particular, FIG. 8 illustrates a flushing procedure of the filter 550 of the filtering connector 316 using a fluid delivery device 860 of the system 700. In some embodiments, the fluid delivery device 860 can include a syringe, a pipet, a catheter, or any other suitable fluid delivery device.

As shown, the fluid delivery device 860 is coupled (e.g., operably coupled, fluidly coupled, etc.) to the injection port 324 of the first branch 212a of the transfer set 202. For example, a needle or another component of the fluid delivery device 860 can be inserted into the injection port 324. Using the fluid delivery device 860, fluid 862 (e.g., water, cleaning solution, etc.) can be injected into the first branch 212a of the transfer set 202 via the injection port 324. In turn, at least a portion of the fluid 862 can flow though the filter 550 and/or the filtering connector 316. The flow of the fluid 862 through the filter 550 can be in a reversed or opposite direction than the flow of the solution 510 through the filter 550 in FIGS. 5B and 5C or the solution 710 through the filtering connector 316 in FIG. 7A. For example, the fluid 862 can flow from the second end portion 552b of the filter 550 toward the first end portion 552a. As discussed above with respect to FIGS. 5A-6, the flow of the fluid 862 through the filter 550 in the reversed direction can at least partially “flush,” “unclog,” or otherwise remove contaminants (e.g., the contaminants 510a) or other matters from the filter 550.

In some embodiments, a portion of the fluid 862 injected into the first branch 212a of the transfer set 202 by the fluid delivery device 860 can flow in a direction away from the filtering connector 316, through the one-way valve 318a, and toward the splitter 214. In these embodiments, the portion of the fluid 862 can at least partially remove or flush contaminants from at least a portion of the first branch 212a of transfer set 202 downstream from the injection port 324. As shown in FIG. 8, the clamp 326 included on the third branch 212c of the transfer set 202 can be placed in a closed state or configuration such that the portion of the fluid 862 that passes through the one-way valve 318a is at least partially blocked or otherwise prevented from reaching the catheter 709. In turn, the portion of the fluid 862 can be redirected into the second branch 212b of the transfer set 202 and can exit the transfer set 202 (e.g., into the drain bag 706) via the connector 320a. Accordingly, in at least some embodiments, the fluid 862 can be used to at least partially remove or flush contaminants from at least a portion of the first branch 212a downstream from the injection port 324 and from the second branch 212b of the transfer set 202.

In other embodiments, the clamp 326 can be place in an open state or configuration during the flushing procedure illustrated in FIG. 8. In these embodiments, the fluid 862 can be inserted into the first branch 212a of the transfer set while solution is drained from a patient via the catheter 709 (e.g., while the solution 717 is drained into the drain bag 706 via the catheter 709, as shown in FIG. 7B). Alternatively, the transfer set 202 can be uncoupled from the catheter 709 before injecting the fluid 862 into the transfer set 202. Continuing with this example, the fluid 862 can be inserted into the first branch 212a of the transfer set 202, and a portion of the fluid 862 can flow into the third branch 212c and exit the transfer set 202 via the connector 320b. In these embodiments, the fluid 862 can be used to at least partially remove or flush contaminants from the third branch 212c of the transfer set 202.

In embodiments in which the first branch 212a includes a clamp (e.g., a clamp similar to the clamp 326 of FIG. 3 and/or positioned between the injection port 324 and the one-way valve 318a), the clamp in the first branch 212a can be placed in a closed state or configuration during the flushing procedure. When the clamp is in the closed state or configuration, a portion of the fluid 862 that is injected into the first branch 212a of the transfer set 202 via the injection port 324 can be at least partially blocked or otherwise prevented from reaching the one-way valve 318a (or other components of the transfer set 202, such as the splitter 214, the second branch 212b, and/or the third branch 212c). In turn, the portion of the fluid 862 can be redirected toward the filter 550 and can exit the transfer set 202 via the filtering connector 316.

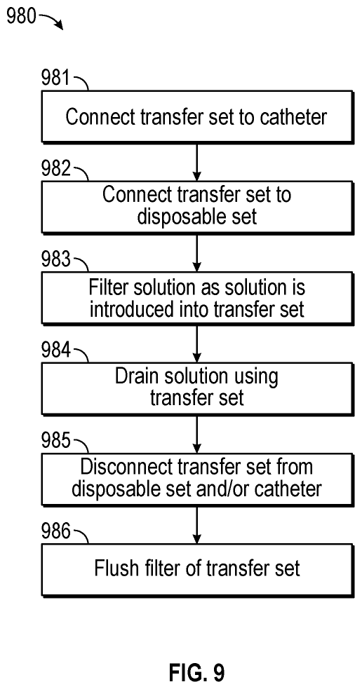

FIG. 9 is a flow diagram illustrating a method 980 of operating a PD system including a transfer set configured in accordance with various embodiments of the present technology. The method 980 is illustrated as a set of blocks, steps, operations, or processes 981-986. All or a subset of the blocks 981-986 can be executed at least in part by various components of a system, such as the PD system 100 of FIG. 1, the PD system 200 of FIG. 2, and/or the PD system 700 of FIGS. 7A-8. For example, all or a subset of the blocks 981-986 can be executed at least in part by a transfer set, a pump, an APD machine, a CAPD stand or assembly, a cassette, a source bag, a drain bag, a catheter, fluid lines, a fluid delivery device, and/or other portions of a disposable set. Additionally, or alternatively, all or a subset of the blocks 981-986 can be executed at least in part by an operator (e.g., a user, a patient, a caregiver, a family member, a physician, etc.) of the system. Furthermore, any one or more of the blocks 981-986 can be executed in accordance with the discussion above. Many of the blocks 981-986 of the method 980 are discussed in detail below with reference to FIGS. 1-8 for the sake of clarity and understanding. It will be appreciated, however, that the method 980 may be used with other suitable PD systems in addition to those described herein.

The method 980 begins at block 981 by connecting a transfer set to a catheter. In some embodiments, for example, connecting the transfer set to the catheter can include connecting a branch of the transfer set to the catheter. The branch can be similar to the branches discussed above with respect to FIGS. 2-8. For example, connecting a transfer set to a catheter can include connecting a third branch 212c of a transfer set 202 to a catheter. Continuing with this example, connecting the transfer set to the catheter can include (a) connecting the third branch 212c of the transfer set 202 to a catheter using a connector 320b and/or (b) forming an impermeable or hermetic seal between the connector 320b and a corresponding connector or adapter of the catheter.

At block 982, the method 980 continues by connecting the transfer set to a disposable set. In some embodiments, connecting the transfer set to a disposable set includes coupling a branch of the transfer set to a source bag. The branch can be similar to the branches discussed above with respect to FIGS. 2-8. For example, coupling the transfer set to the source bag can include coupling a first branch 212a of a transfer set 202 to a source bag. Continuing with this example, coupling the first branch 212a of the transfer set 202 to a source bag can include (a) connecting a filtering connector 316 of the first branch 212a to a corresponding connector of the source bag or to a corresponding connector of a fluid line coupled to the source bag, and/or (b) forming an impermeable or hermetic seal between the filtering connector 316 and the corresponding connector.

In these and other embodiments, connecting the transfer set to a disposable set includes coupling a branch of the transfer set to a drain bag. The branch can be similar to the branches discussed above with respect to FIGS. 2-8. For example, coupling the transfer set to the drain bag can include coupling a second branch 212b of a transfer set 202 to a drain bag. Continuing with this example, coupling the second branch 212b of the transfer set 202 to a drain bag can include (a) connecting a connector 320a of the second branch 212b to a corresponding connector of the drain bag or to a corresponding connector of a fluid line coupled to the drain bag, and/or (b) forming an impermeable or hermetic seal between the connector 320a and the corresponding connector.

At block 983, the method 980 continues by filtering solution introduced into the transfer set. For example, filtering solution introduced into the transfer set can include filtering solution received from the source bag coupled to the transfer set. Continuing with this example, filtering the solution received from the source bag can include receiving the solution via a filtering connector of the transfer set, such as the filtering connector 316 described above with respect to block 982. In these and other embodiments, filtering the solution can include flowing the solution through a filter having one or more filtering elements, such as the filter 550 of the filtering connector 316 discussed above with respect to FIGS. 5B-6. Flowing the solution through the filter can include removing (e.g., separating, filtering, etc.) contaminants or other matters from the solution using the filter. In these and still other embodiments, filtering the solution can include (a) permitting the solution to flow through the filter and into the catheter coupled to the transfer set (e.g., for introduction of the solution into a patient), and/or (b) prevent the contaminants or other matters from flowing through the filter or into the catheter. In these and other embodiments, filtering the solution can include at least partially closing a clamp of the transfer set (e.g., placing the clamp in a closed state or configuration), such as a clamp positioned on the second branch of the transfer set.

At block 984, the method 980 continues by draining solution using the transfer set. In some embodiments, draining the solution using the transfer set can include draining waste solution from a patient. In these and other embodiments, draining the solution using the transfer set includes draining the solution into the drain bag coupled to the transfer set. In some embodiments, draining the solution can include preventing the solution from contacting or otherwise interacting with various components of the transfer set, such as the filtering connector and/or using one or more one-way valves or clamps.

At block 985, the method 980 continues by at least partially disconnecting the transfer set from the disposable set and/or from the catheter. In some embodiments, at least partially disconnecting the transfer set from the disposable set includes disconnecting the transfer set from the source bag and/or from the drain bag. Disconnecting the transfer set from the disposable set and/or from the catheter can include uncoupling the filtering connector and/or the connector(s) of the first branch, the second branch, and/or the third branch of the transfer set from the corresponding connectors of the source bag (or of a fluid line coupled to the source bag), the drain bag (or of a fluid line coupled to the drain bag), and/or the catheter, respectively.

At block 986, the method 980 continues by flushing a filter of the transfer set. In some embodiments, flushing the filter can flushing the filter using an injection port of the transfer set and/or a fluid delivery device. For example, flushing the filter can include injecting fluid into the transfer set via the injection port and a fluid delivery device coupled to the injection port. Flushing the filter can include flowing fluid (e.g., injected into the transfer set) toward the filtering connector and/or a filter of the transfer set (e.g., from a second end portion of the filter to a first end portion of the filter). Flushing the filter can include flushing contaminants and/or other matters out of the filter (e.g., filtering elements) using the fluid. In these and other embodiments, flushing the filter can include at least partially clamping or closing a clamp of the transfer set, such as a clamp positioned on the third branch of the transfer set and/or a clamp positioned on the first branch of the transfer set. In these and still other embodiments, flushing the filter includes flowing fluid injected into the transfer set through the first branch of the transfer set, through the second branch of the transfer set, and/or into the drain bag coupled to the transfer set.

Although the steps of the method 980 are discussed and illustrated in a particular order, the method 980 illustrated in FIG. 9 is not so limited. In other embodiments, the method 980 can be performed in a different order. In these and other embodiments, any of the steps of the method 980 (e.g., block 982) can be performed before, during, and/or after any of the other steps of the method 980 (e.g., block 981). Moreover, a person of ordinary skill in the relevant art will recognize that the illustrated method 980 can be altered and still remain within these and other embodiments of the present technology. For example, one or more steps of the method 980 (e.g., block 985) illustrated in FIG. 9 can be omitted and/or repeated in some embodiments.

### C. EXAMPLES

Several aspects of the present technology are set forth in the following examples. Although several aspects of the present technology are set forth in examples specifically directed to apparatuses, systems, and methods; any of these aspects of the present technology can similarly be set forth in examples directed to any of devices/apparatuses, systems, methods, and computer-readable mediums in other embodiments.
1. A transfer set, comprising:
a first connector configured to be coupled to a disposable set of a peritoneal dialysis (PD) system;
a second connector configured to be coupled to a catheter of the PD system;
a fluid channel extending between the first connector and the second connector; and
a filter positioned within the fluid channel and configured to filter contaminants from solution flowing within the fluid channel between the first connector and the second connector.
2. The transfer set of example 1 wherein the first connector includes the filter.
3. The transfer set of example 1 or example 2 wherein the filter includes:
a first end portion;
a second end portion opposite the first end portion; and
a filtering element having a first end and a second end opposite the first end, wherein the filtering element extends at least partway between the first end portion and the second end portion of the filter, and wherein the first end and the second end of the filtering element are both positioned at the first end portion of the filter.
4. The transfer set of example 3 wherein:
the filtering element includes a semi-permeable membrane;
the filtering element is configured to receive the solution and the contaminants into an interior of the filtering element via the first end or the second end; and
the filtering element is configured to (a) permit the solution to flow through the semi-permeable membrane and continue flowing toward the second end portion of the filter and (b) prevent the contaminants from flowing through the semi-permeable membrane.
5. The transfer set of any of examples 1-4 wherein the filter is an ultrafilter.
6. The transfer set of any of examples 1-5, further comprising a one-way valve (i) positioned between the filter and the second connector, (ii) configured to permit a fluid to flow through the one-way valve in a first direction toward the second connector, and (iii) prevent the fluid from flowing through the one-way valve in a second direction toward the filter.
7. The transfer set of any of examples 1-6, further comprising an injection port positioned within the fluid channel and configured to receive a fluid from a fluid delivery device when the fluid delivery device is removably coupled to the injection port.
8. The transfer set of example 7, further comprising a one-way valve within the fluid channel and positioned between the filter and the second connector, wherein the injection port is positioned between the filter and the one-way valve.
9. The transfer set of any of examples 1-8 wherein:
the fluid channel is a first fluid channel;
the transfer set includes a first branch and a second branch:
the first branch includes the first connector, the filter, and a first portion of the first fluid channel;
the second branch includes the second connector and a second portion of the first fluid channel;
the transfer set further includes:
   a third connector configured to be coupled to the disposable set of the PD system, and
   a third branch including the third connector and a second fluid channel; and
the first branch, the second branch, and the third branch are connected to one another such that the first portion of the first fluid channel, the second portion of the first fluid channel, and the second fluid channel are fluidly coupled to one another.
10. The transfer set of example 9, further comprising a one-way valve positioned within the second fluid channel and configured to prevent a fluid from flowing through the one-way valve away from the third connector.
11. The transfer set of any of examples 1-10, further comprising a clamp positioned between the filter and the second connector, wherein the clamp has (a) an open configuration in which a fluid is permitted to flow in a first direction through the clamp toward the second connector and (b) a closed configuration in which the fluid is prevented from flowing in the first direction and in a second direction through the clamp toward the filter.
12. A peritoneal dialysis (PD) system, comprising:
a disposable set including (a) a source bag configured to dispense a first solution and (b) a drain bag configured to receive a second solution;
a catheter configured to be at least partially installed in a peritoneal space of a patient; and
a transfer set configured to transfer (i) the first solution received from the source bag to the catheter and (ii) the second solution received from the catheter toward the drain bag, wherein the transfer set includes—
   a first branch (i) fluidly coupled to the source bag and (ii) having a filter configured to filter contaminants from the first solution received from the source bag;
   a second branch (i) fluidly coupled to the drain bag and (ii) configured to transfer the second solution toward the drain bag; and
   a third branch (i) fluidly coupled to the catheter, (ii) configured to transfer the first solution from the first branch to the catheter, and (iii) configured to transfer the second solution from the catheter to the second branch.
13. The PD system of example 12 wherein:
the first branch further includes a first connector fluidly coupling the first branch to the source bag; and
the first connector includes the filter.
14. The PD system of example 12 or example 13 wherein the first branch further includes a first flow-biasing element (i) positioned between the filter and the third branch and (ii) configured to prevent the first solution or the second solution from flowing through the first flow-biasing element toward the filter.
15. The PD system of any of examples 12-14 wherein the second branch includes a second flow-biasing element configured to prevent the second solution from flowing through the second flow-biasing element toward the first branch or the third branch.
16. The PD system of any of examples 12-15 wherein the first branch further includes an injection port (a) positioned between the filter and the third branch and (b) configured to receive a fluid from a fluid delivery device (i) when the fluid delivery device is removably coupled to the injection port and (ii) such that at least a portion of the fluid flows toward the filter and flushes contaminants from the filter.
17. A method of operating a transfer set, the method comprising:
receiving, via an opening in a first end portion of the transfer set, a solution from a source bag of a disposable set of a peritoneal dialysis (PD) system, the source bag fluidly coupled to the first end portion of the transfer set;
filtering the solution using a filter of the transfer set positioned along the first end portion of the transfer set; and
transferring, via the first end portion of the transfer set and a second end portion of the transfer set, the solution to a catheter fluidly coupled to the second end portion of the transfer set.
18. The method of example 17 wherein filtering the solution includes applying a hydrostatic pressure across the filter in a direction toward the second end portion of the transfer set.
19. The method of example 17 or example 18, further comprising flushing the filter of the transfer set using a fluid flowing through at least a portion of the first end portion of the transfer set.
20. The method of example 19 wherein flushing the filter includes receiving the fluid via an injection port of the transfer set positioned along the first end portion of the transfer set, wherein the injection port is different from the opening in the first end portion of the transfer set.
21. The method of example 19 or example 20 wherein flushing the filter includes applying a hydrostatic pressure across the filter in a direction toward the opening in the first end portion of the transfer set.
22. The method of any of examples 17-21 wherein:
the opening in the first end portion of the transfer set is a first opening;
the solution is a first solution;
receiving, via a second opening in the second end portion of the transfer set, a second solution from the catheter; and
transferring, via the second end portion of the transfer set and a third opening in a third end portion of the transfer set, the second solution toward a drain bag of the disposable set of the PD system, the drain bag fluidly coupled to the third end portion of the transfer set.
23. The method of example 22 wherein transferring the second solution toward the drain bag includes preventing, using a flow-biasing element positioned along the first end portion of the transfer set, at least a portion of the second solution from flowing along at least a portion of the first end portion of the transfer set in a direction toward the source bag.
24. The method of example 22 or example 23 wherein transferring the second solution toward the drain bag includes preventing, using a flow-biasing element positioned along the third end portion of the transfer set, at least a portion of the second solution from flowing in a direction away from the drain bag and toward the catheter first end portion or the second end portion of the transfer set.

### D. CONCLUSION

From the foregoing, it will be appreciated that specific embodiments of the technology have been described herein for purposes of illustration, but well-known structures and functions have not been shown or described in detail to avoid unnecessarily obscuring the description of the embodiments of the technology. To the extent any materials incorporated herein by reference conflict with the present disclosure, the present disclosure controls. Where the context permits, singular or plural terms can also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and both A and B. Where the context penults, singular or plural terms can also include the plural or singular term, respectively. Additionally, the terms “comprising,” “including,” “having” and “with” are used throughout to mean including at least the recited feature(s) such that any greater number of the same feature and/or additional types of other features are not precluded.

Furthermore, as used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. Moreover, the terms “connect” and “couple” are used interchangeably herein and refer to both direct and indirect connections or couplings. For example, where the context permits, element A “connected” or “coupled” to element B can refer (i) to A directly “connected” or directly “coupled” to B and/or (ii) to A indirectly “connected” or indirectly “coupled” to B.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments can perform steps in a different order. As another example, various components of the technology can be further divided into subcomponents, and/or various components and/or functions of the technology can be combined and/or integrated. Furthermore, although advantages associated with certain embodiments of the technology have been described in the context of those embodiments, other embodiments can also exhibit such advantages, and not all embodiments need necessarily exhibit such advantages to fall within the scope of the technology.

It should also be noted that other embodiments in addition to those disclosed herein are within the scope of the present technology. For example, embodiments of the present technology can have different configurations, components, and/or procedures in addition to those shown or described herein. Moreover, a person of ordinary skill in the art will understand that these and other embodiments can be without several of the configurations, components, and/or procedures shown or described herein without deviating from the present technology. Accordingly, the disclosure and associated technology can encompass other embodiments not expressly shown or described herein.

1. A transfer set, comprising:
a first connector configured to be coupled to a disposable set of a peritoneal dialysis (PD) system;
a second connector configured to be coupled to a catheter of the PD system;
a fluid channel extending between the first connector and the second connector; and
a filter positioned within the fluid channel and configured to filter contaminants from solution flowing within the fluid channel between the first connector and the second connector.

2. The transfer set of claim 1, wherein the first connector includes the filter.

3. The transfer set of claim 1, wherein the filter includes:
a first end portion;
a second end portion opposite the first end portion; and
a filtering element having a first end and a second end opposite the first end, wherein the filtering element extends at least partway between the first end portion and the second end portion of the filter, and wherein the first end and the second end of the filtering element are both positioned at the first end portion of the filter.

4. The transfer set of claim 3, wherein:
the filtering element includes a semi-permeable membrane;
the filtering element is configured to receive the solution and the contaminants into an interior of the filtering element via the first end or the second end; and
the filtering element is configured to (a) permit the solution to flow through the semi-permeable membrane and continue flowing toward the second end portion of the filter and (b) prevent the contaminants from flowing through the semi-permeable membrane.

5. The transfer set of claim 1, wherein the filter is an ultrafilter.

6. The transfer set of claim 1, further comprising a one-way valve (i) positioned between the filter and the second connector, (ii) configured to permit a fluid to flow through the one-way valve in a first direction toward the second connector, and (iii) preventing the fluid from flowing through the one-way valve in a second direction toward the filter.

7. The transfer set of claim 1, further comprising an injection port positioned within the fluid channel and configured to receive a fluid from a fluid delivery device when the fluid delivery device is removably coupled to the injection port.

8. The transfer set of claim 7, further comprising a one-way valve within the fluid channel and positioned between the filter and the second connector, wherein the injection port is positioned between the filter and the one-way valve.

9. The transfer set of claim 1, wherein:
the fluid channel is a first fluid channel;
the transfer set includes a first branch and a second branch;
the first branch includes the first connector, the filter, and a first portion of the first fluid channel;
the second branch includes the second connector and a second portion of the first fluid channel;
the transfer set further includes:
a third connector configured to be coupled to the disposable set of the PD system, and
a third branch including the third connector and a second fluid channel; and
the first branch, the second branch, and the third branch are connected to one another such that the first portion of the first fluid channel, the second portion of the first fluid channel, and the second fluid channel are fluidly coupled to one another.

10. The transfer set of claim 9, further comprising a one-way valve positioned within the second fluid channel and configured to prevent a fluid from flowing through the one-way valve away from the third connector.

11. The transfer set of claim 1, further comprising a clamp positioned between the filter and the second connector, wherein the clamp has (a) an open configuration in which a fluid is permitted to flow in a first direction through the clamp toward the second connector and (b) a closed configuration in which the fluid is prevented from flowing in the first direction and in a second direction through the clamp toward the filter.

12. A peritoneal dialysis (PD) system, comprising:
a disposable set including (a) a source bag configured to dispense a first solution and (b) a drain bag configured to receive a second solution;
a catheter configured to be at least partially installed in a peritoneal space of a patient; and
a transfer set configured to transfer (i) the first solution received from the source bag to the catheter and (ii) the second solution received from the catheter toward the drain bag, wherein the transfer set includes:
a first branch (i) fluidly coupled to the source bag and (ii) having a filter configured to filter contaminants from the first solution received from the source bag;
a second branch (i) fluidly coupled to the drain bag and (ii) configured to transfer the second solution toward the drain bag; and
a third branch (i) fluidly coupled to the catheter, (ii) configured to transfer the first solution from the first branch to the catheter, and (iii) configured to transfer the second solution from the catheter to the second branch.

13. The PD system of claim 12, wherein:
the first branch further includes a first connector fluidly coupling the first branch to the source bag; and
the first connector includes the filter.

14. The PD system of claim 12, wherein the first branch further includes a first flow-biasing element (i) positioned between the filter and the third branch and (ii) configured to prevent the first solution or the second solution from flowing through the first flow-biasing element toward the filter.

15. The PD system of claim 12, wherein the second branch includes a second flow-biasing element configured to prevent the second solution from flowing through the second flow-biasing element toward the first branch or the third branch.

16. The PD system of claim 12, wherein the first branch further includes an injection port (a) positioned between the filter and the third branch and (b) configured to receive a fluid from a fluid delivery device (i) when the fluid delivery device is removably coupled to the injection port and (ii) such that at least a portion of the fluid flows toward the filter and flushes contaminants from the filter.

17. A method of operating a transfer set, the method comprising:
receiving, via an opening in a first end portion of the transfer set, a solution from a source bag of a disposable set of a peritoneal dialysis (PD) system, the source bag fluidly coupled to the first end portion of the transfer set;
filtering the solution using a filter of the transfer set positioned along the first end portion of the transfer set; and
transferring, via the first end portion of the transfer set and a second end portion of the transfer set, the solution to a catheter fluidly coupled to the second end portion of the transfer set.

18. The method of claim 17, wherein filtering the solution includes applying a hydrostatic pressure across the filter in a direction toward the second end portion of the transfer set.

19. The method of claim 17, further comprising flushing the filter of the transfer set using a fluid flowing through at least a portion of the first end portion of the transfer set.

20. The method of claim 19, wherein flushing the filter includes receiving the fluid via an injection port of the transfer set positioned along the first end portion of the transfer set, wherein the injection port is different from the opening in the first end portion of the transfer set.

21. The method of claim 19, wherein flushing the filter includes applying a hydrostatic pressure across the filter in a direction toward the opening in the first end portion of the transfer set.

22. The method of claim 17, wherein:
the opening in the first end portion of the transfer set is a first opening;
the solution is a first solution;
receiving, via a second opening in the second end portion of the transfer set, a second solution from the catheter; and
transferring, via the second end portion of the transfer set and a third opening in a third end portion of the transfer set, the second solution toward a drain bag of the disposable set of the PD system, the drain bag fluidly coupled to the third end portion of the transfer set.

23. The method of claim 22, wherein transferring the second solution toward the drain bag includes preventing, using a flow-biasing element positioned along the first end portion of the transfer set, at least a portion of the second solution from flowing along at least a portion of the first end portion of the transfer set in a direction toward the source bag.

24. The method of claim 22, wherein transferring the second solution toward the drain bag includes preventing, using a flow-biasing element positioned along the third end portion of the transfer set, at least a portion of the second solution from flowing in a direction away from the drain bag and toward the catheter first end portion or the second end portion of the transfer set.
